(19) United States
(12) Patent Application Publication
(10) Pub. No.: US 2019/0031617 A1
(43) Pub. Date: Jan. 31, 2019

(54) PROCESS FOR THE MANUFACTURE OF CARBOXAMIDES

(71) Applicant: SOLVAY SA, Brussels (BE)

(72) Inventor: Suzanne WASSMANN, Hannover (DE)

(21) Appl. No.: 16/077,561

(22) PCT Filed: Feb. 10, 2017
(86) PCT No.: PCT/EP2017/053027

§ 371 Date: Aug. 13, 2018

(30) Foreign Application Priority Data
Feb. 18, 2016 (EP) 16156331.7

(51) Int. Cl. C07D 231/44; A01N 43/56

(52) U.S. Cl. CPC C07D 231/44; A01N 43/56

(57) ABSTRACT

The present invention concerns a process for the manufacture of carboxamides, in particular agrochemical or pharmaceutically active ingredients, from pyrazole ketone compounds.

This application claims priority to European application 16156331.7, the whole content of this application being incorporated herein by reference for all purposes.

The present invention concerns a process for the manufacture of carboxamides, in particular agrochemical or pharmaceutically active ingredients, from pyrazole ketone compounds.

Agrochemical and pharmaceutical active ingredients which contain a carboxamide moiety, in particular a pyrazole building block bound to the residual molecule by a carboxamide moiety, have gained importance over the last years. SDHI (Succinatedehydrogenase inhibitor) fungicides, including for example, Sedaxane, Bixafen or Benzovindiflupyr, are among the recently developed agrochemical active ingredients comprising such an entity. For the manufacture of such compounds, regularly 3-halomethylpyrazole-4-yl carboxylic acids, often obtained by hydrolysis of their esters, are converted into the carboxamides, for example after conversion into the 3-halomethylpyrazole-4-yl carboxylic acid halide.

The invention concerns a process for the manufacture of carboxamides, comprising the step of reacting a pyrazole compound of the formula (I)

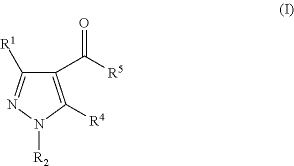

with a compound of the formula (II) NR³H(A*)Q to manufacture a compound of the formula (III)

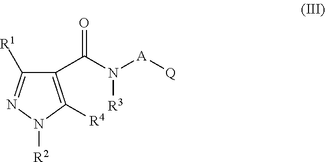

wherein R¹ is selected from the group consisting of H, R′, X′, CN, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted, and X′ is selected from the group F, Cl, Br and I;

wherein R² is selected from the group consisting of optionally substituted C₁-C₁₂-alkyl, C₂-C₆ alkenyl, cycloalkyl, aryl, heteroaryl and aralkyl; wherein R³ is selected from the group consisting of H, C₁-C₁₂-alkyl, C₂-C₆ alkenyl or C₃-C₈-cycloalkyl group, each of which is optionally substituted;

wherein R⁴ is selected from the group consisting of H, X′, COOR′, OR′, SR′, C(O)NR′₂, wherein R′ and X′ are defined above;

wherein R⁵ is selected from the group consisting of C₁-C₁₂-alkyl, C₂-C₆ alkenyl, cycloalkyl, aryl, heteroaryl and aralkyl, each of which are optionally substituted;

wherein A* is absent or an optionally substituted C₁-C₄-alkylene group;

wherein Q is an optionally substituted aryl or heteroaryl group.

The invention also concerns a process for the manufacture of an agrochemically or pharmaceutically active compound, which comprises the process of anyone of reacting a pyrazole compound of formula (I) with a compound of the formula (II) NR³H(A*)Q to obtain a compound of the formula (III). Another object of the present invention is the use of a compound of formula (I) for the manufacture of an agrochemically or pharmaceutically active compound, wherein (I) is reacted with a compound of formula (II).

In the present invention, designations in singular are in intended to include the plural; for example, “a solvent” is intended to denote also “more than one solvent” or “a plurality of solvents”.

In the context of the present invention, the term “comprising” is intended to include the meaning of “consisting of”.

When a double bond is depicted in a particular E/Z geometry, this is intended to also denote the other geometric form as well as mixtures thereof.

For the purpose of the present invention, the definition C₁-C₁₂-alkyl comprises the largest range defined herein for an alkyl group. Specifically, this definition comprises, for example, the meanings methyl, ethyl, n-propyl, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl and n-dodecyl. Often, methyl, ethyl, n-propyl, isopropyl, n-, iso-, sec- and t-butyl are most preferred residues selected from the group C₁-C₁₂-alkyl.

The term “C₃-C₁₀-cycloalkyl”, as used in this invention, denotes mono-, bi- or tricyclic hydrocarbon groups comprising 3 to 10 carbon atoms, especially 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl.

The term “C₂-C₆-alkenyl group” denotes a group comprising a carbon chain and at least one double bond. Alkenyl groups are, for example, ethenyl, propenyl, butenyl, pentenyl or hexenyl.

In the context of the present invention, aryl groups are, unless defined otherwise, aromatic hydrocarbon groups comprising the largest range of C₅-C₁₈ carbon atoms and which may optionally be substituted by further groups selected from the group consisting of R′, —X′, —OR′, —SR′, —NR′₂, —SiR′₃, —COOR′, —(C—O)R′, —CN and —CONR′₂, where R′ and X′ are defined as above. Specifically, the term “aryl groups” comprises, for example, the meanings cyclopentadienyl, phenyl, cycloheptatrienyl, cyclooctatetraenyl, naphthyl and anthracenyl.

The term “heteroaryl” denotes an aromatic cyclic radical containing 3 to 6 ring atoms, of which 1 to 4 are from the group of oxygen, nitrogen and sulfur, and which may additionally be fused by a benzo ring, and which may optionally be substituted by further groups selected from the group consisting of R′, —X′, —OR′, —SR′, —NR′₂, —SiR′₃, —COOR′, —(C—O)R′, —CN and —CONR′₂, where R′ and X′ are defined as above. For example, heteroaryl is benzimidazol-2-yl, furanyl, imidazolyl, isoxazolyl, isothiazolyl, oxazolyl, pyrazinyl, pyrimidinyl, pyridazinyl, pyridinyl, benzisoxazolyl, thiazolyl, pyrrolyl, pyrazolyl, thiophenyl, 1,2,3-oxadiazolyl, 1,2,4-oxadiazolyl, 1,2,5-oxadiazolyl, 1,3,4-oxadiazolyl, 1,2,4-triazolyl, 1,2,3-triazolyl, 1,2,5-triazolyl, 1,3,4-triazolyl, 1,2,4-triazolyl, 1,2,4-thiadiazolyl, 1,3,4-thiadiazolyl, 1,2,3-thiadiazolyl, 1,2,5-thiadiazolyl, 2H-1,2,3,4-tetrazolyl, 1H-1,2,3,4-tetrazolyl, 1,2,3,4-oxatriazolyl, 1,2,3,5-oxatriazolyl, 1,2,3,4-thiatriazolyl and 1,2,3,5-thiatriazolyl.

In the context of the present invention, arylalkyl or heteroarylalkyl groups (aralkyl or heteroaralkyl groups) are, unless defined otherwise, alkyl groups which are substituted by aryl or heteroaryl groups, which are defined as above, which may have a C₁₋₈-alkylene chain and which may be substituted in the alkylene chain by one or more heteroatoms selected from the group consisting of O, N, P and S and which can optionally be substituted by further groups selected from the group consisting of R′, —X′, —OR′, —SR′, —NR′₂, —SiR′₃, —COOR′, —(C—O)R′, —CN and —CONR′₂, where R′, and X′ are defined as above.

The definition C₇-C₁₉-aralkyl or C₇-C₁₉-heteroaralkyl group comprises the largest range defined herein for an arylalkyl or heteroaralkyl group having a total of 7 to 19 atoms in the skeleton and the alkylene chain. Specifically, this definition comprises, for example, the meanings benzyl and phenylethyl.

In the context of the present invention, alkylaryl or alkylheteroaryl groups (alkaryl or alkheteroaryl groups) are, unless defined otherwise, aryl or heteroaryl groups which are substituted by alkyl groups, which may have a C₁-C₈-alkylene chain and which may be substituted in the alkylene chain by one or more heteroatoms selected from the groups consisting of O, N, P and S and optionally by further groups selected from the group consisting of R′, —X′, —OR′, —SR′, —NR′₂, —SiR′₃, —COOR′, —(C—O)R′, —CN and —CONR′₂, where R′, and X′ are defined as above.

The definition C7-C19-alkylaryl and C7-C19-alkylheteroaryl group comprises the largest range defined herein for an alkylaryl or alkylheteroaryl group having a total of 7 to 19 atoms in the skeleton and the alkylene chain. Specifically, this definition comprises, for example, the meanings tolyl, 2,3-, 2,4-, 2,5-, 2,6-, 3,4- or 3,5-dimethylphenyl.

R¹ is selected from the group consisting of H, R′, X′, CN, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted by one or more groups selected from the group consisting of —R″, —X′, —OR″, —SR″, —NR″ ₂, —SiR″₃, —COOR″, —CN and —CONR″₂, where R″ independently is hydrogen or a C₁-C₁₂-alkyl group, and X′ is selected from the group F, Cl, Br and I. In one embodiment, R¹ is an optionally substituted C₁-C₁₂-alkyl, C₁-C₁₂-alkyl, C₂-C₆ alkenyl, aryl, cycloalkyl, aralkyl and heteroaryl. In one aspect, R¹ is a C₁-C₄ alkyl, which is the group consisting of methyl, ethyl, i-propyl, n-propyl, n-butyl, tert-butyl, iso-butyl and sec-butyl. Preferably, the C₁ -C₄ group is substituted by one or more X′, wherein X′ is independently selected from F, Cl, Br and I. More preferably, R¹ is selected from the group consisting of CF₂Cl, CF₂H, CFCl₂, CFClH, CF₂Br, CCl₃, CF₃, CBr₃, and CI₃. Even more preferably, R¹ is selected from the group consisting of CF₂Cl, CF₂H and CF₃. In one very preferred aspect, R¹ is CHF₂. In another very preferred aspect, R¹ is CF₃.

In one embodiment, R¹ is an optionally substituted cycloalkyl, preferably selected from the group consisting of include cyclopropyl, cyclobutyl, cyclopentyl, cyclohexyl, cycloheptyl and cyclooctyl.

R² is selected from the group consisting of optionally substituted C₁⁻C₁₂-alkyl, C₂-C₆ alkenyl, cycloalkyl, aryl, heteroaryl and aralkyl. Each of R² optionally is substituted by one or more groups selected from the group consisting of H, R′, X′, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted by one or more groups selected from the group consisting of —R″, —X′, —OR″, —SR″, —NR″₂, 'SiR″₃, —COOR″, —CN and —CONR″₂, where R″ independently is hydrogen or a C₁-C₁₂-alkyl group, and X′ is selected from the group F, Cl, Br and I. In one aspect, R² is H or an optionally substituted C₁-C₄-alkyl group. Preferably, R² is optionally substituted methyl. More preferably, R² is CH₃.

R³ is selected from the group consisting of H, C₁-C₁₂-alkyl, C₂-C₆ alkenyl or C₃-C₈-cycloalkyl group, each of which is optionally substituted by one or more group independently selected from the group consisting of R′, X′, CN, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted by one or more groups selected from the group consisting of —R″, —X′, —OR″, —SR″, —NR″₂, —SiR″₃, —COOR″, —CN and —CONR″₂, where R″ independently is hydrogen or a C₁-C₁₂-alkyl group, and X′ is selected from the group F, Cl, Br and I. In one aspect, R³ preferably is selected from H and optionally substituted C₁-C₄-alkyl. In a more preferred aspect, R³ is CH₃ or H. Most preferably, R³ is H.

R⁴ is selected from the group consisting of H, X′, COOR′, OR′, SR′, C(O)NR′₂, wherein R′ and X′ are defined above. Preferably, R⁴ is selected from H, an optionally substituted C₁-C₄-alkyl group selected from methyl, ethyl, i-propyl, n-propyl, n-butyl, tert-butyl, iso-butyl and sec-butyl, or X′. When R⁴ is X′, R⁴ preferably is F or Cl. Preferably, R⁴is H or F.

R⁵ is selected from the group consisting of C₁-C₁₂-alkyl, C₂-C₆ alkenyl, cycloalkyl, aryl, heteroaryl, aralkyl, each of which is optionally substituted. Each R⁵ can optionally be substituted by one or more groups independently selected from the group consisting of R′, X′, CN, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted by one or more groups selected from the group consisting of —R″, —X′, —OR″, —SR″, —NR″₂, —SiR″₃, —COOR″, —CN and —CONR″₂, where R″ independently is hydrogen or a C₁-C₁₂-alkyl group, and X′ is selected from the group F, Cl, Br and I. Preferably, R⁵ is an alkyl or cycloalkyl residue, each of which is optionally substituted. In one aspect, R⁵ is preferably selected from the group of C₁-C₄-alkyl, which consists specifically of methyl, ethyl, i-propyl, n-propyl, n-butyl, tert-butyl, iso-butyl and sec-butyl. Most preferably, R⁵ is methyl. R⁵ generally is a group which can be removed selectively by cleavage of the bond between R⁵ and the carbonyl carbon atom, preferably by oxidative amination.

In one embodiment, when R⁵ is an alkyl group, in particular an alkyl group selected from the group of C₁-C₄-alkyl defined above, which is substituted by zero, one or more halogen atoms independently selected from X′ as defined above, the total number p of X′ atoms is p≤2n, wherein n is the number of carbon atoms in the alkyl residue.

In another embodiment, R⁵ is selected from the group consisting of CCl₃, CF₃, CBr₃ and CI₃, wherein CCl₃ and CF₃ are preferred. In this embodiment, the reaction between (I) and (II) preferably is carried out in the presence of an oxidation agent, preferably oxygen, and at least one catalyst.

A* is absent or a C₁-C₄-alkylene group which can be substituted by one or more groups selected from the group consisting of H, R′, X′, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted by one or more groups selected from the group consisting of —R″, —X′, —OR″, —SR″, —NR″₂, —SiR″₃, —COOR″, —CN and —CONR″₂, where R″ independently is hydrogen or a C₁-C₁₂-alkyl group, and X′ is selected from the group F, Cl, Br and I. When A* is an, optionally substituted, C₁-C₄-alkylene group, A* can be interrupted by one or more atoms selected from the group consisting of O, N and S. Preferably, A* is absent. In another aspect, A* is selected from —CH₂—, —CH₂—CH₂— and —CH₂—CH₂—CH₂—.

Q is an optionally substituted aryl or heteroaryl group. The aryl or heteroaryl group can also be bi- or tricyclic, wherein one or more rings which are bound to the aryl or heteroaryl group can be non-aromatic. Generally, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thioazole, thiadiazole, oxazole, oxadiazole, pyridine, pyrimidine, triazine, tetrazine, thiazine, azepine and diazepine, each of which is optionally substituted. In a particular aspect, Q is selected from Q1 to Q38 defined here below:

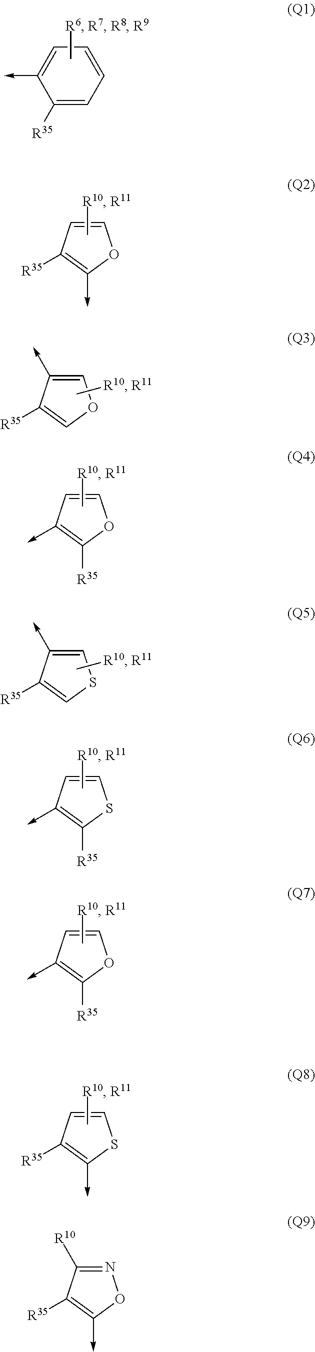

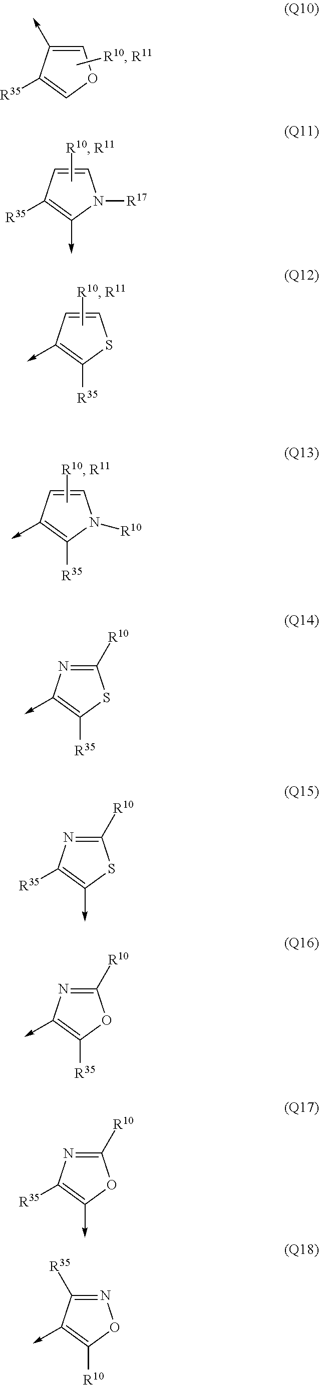

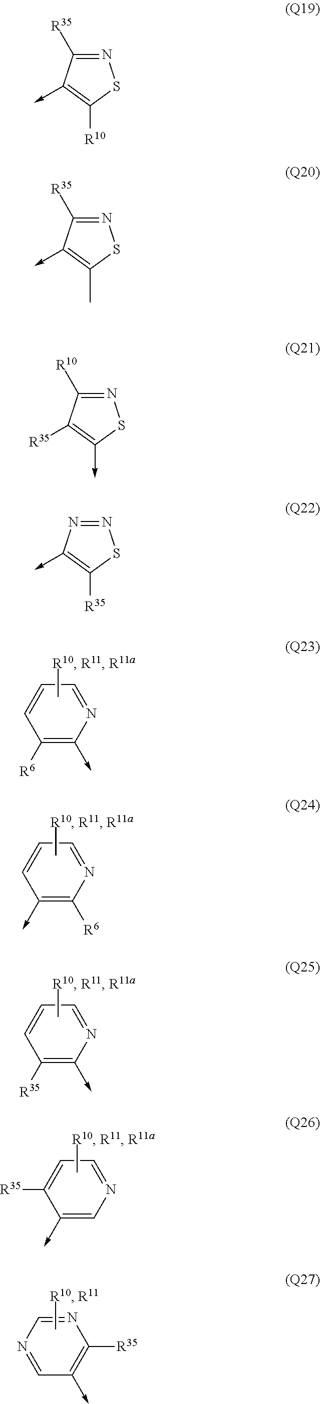

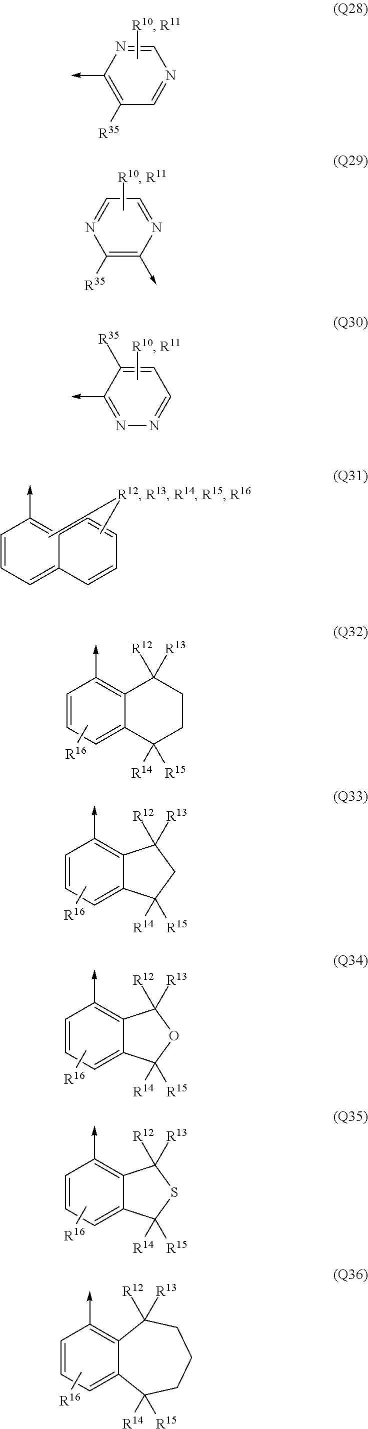

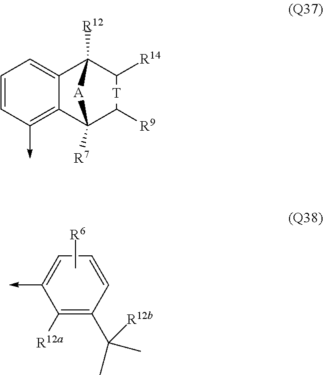

wherein R²⁸ is a hydrogen, C₁₋₁₂ alkyl, C₂₋₁₂ alkenyl or C₂₋₁₂ alkynyl group, which may be substituted by 1 to 6 substituents, each substituent independently selected from halogen, cyano, C₁₋₄ alkoxy, C₁₋₄ thioalkyl, COO—C₁₋₄ alkyl, ═N—OH, ═N—O—(C₁₋₄ alkyl), C₃₋₈ cycloalkyl, which may itself be substituted by 1 to 3 substituents, each independently selected from C₁₋₄ alkyl, halogen, C₁₋₄ alkoxy and C₁₋₄ haloalkoxy, and C₄₋₈ cycloalkenyl, which may itself be substituted by 1 to 3 substituents, each independently selected from C₁₋₄ alkyl, halogen, C₁₋₄ alkoxy and C₁₋₄ haloalkoxy;

or R³⁵ is a C₃₋₈ cycloalkyl, C₄₋₈ cycloalkenyl or C₅₋₈ cycloalkadienyl group, which may be substituted by 1 to 3 substituents, each independently selected from halogen, C₁₋₄ alkyl, C₁₋₄ haloalkyl, C₁₋₄ alkoxy, C_{1- 4} haloalkoxy, C₁-₄ thioalkyl, C₃₋₆ cycloalkyl, which may itself be substituted by 1 to 3 substituents, each independently selected from C₁₋₄ alkyl, halogen, C₁₋₄ alkoxy and C₁₋₄ haloalkoxy, and phenyl, which may itself be substituted by 1 to 5 independently selected halogen atoms;

or R³⁵ is a C₆₋₁₂ bicycloalkyl, C₆₋₁₂ bicycloalkenyl or C₆₋₁₂ bicycloalkadienyl group, which may be substituted by 1 to 3 substituents, each independently selected from halogen, C₁₋₄ alkyl and C₁₋₄ haloalkyl;

or R³⁵ is phenyl, which may be substituted by 1 to 3 substituents, each independently selected from halogen, cyano, nitro, C₁₋₄ alkyl, C₁₋₄ haloalkyl, C₁₋₄ alkoxy, C₁₋₄ alkylthio, C₁₋₄ haloalkoxy, C₁₋₄ haloalkylthio, C(H)═N—OH, C(H)═N—O(C₁₋₆ alkyl), C(C₁₋₆ alkyl)═N—OH, C(C₁₋₆ alkyl)═N—O—(C₁₋₆ alkyl), (E)C≡CR, (E)ₙCR²⁷═CR²⁵ R²⁶, phenyl, which may itself be substituted by 1 to 3 substituents, each independently selected from halogen, cyano, nitro, C₁₋₄ alkyl, C₁₋₄ haloalkyl, C₁₋₄ alkoxy, C₁₋₄ haloalkoxy, C₁₋₄ haloalkylthio, C(H)═N—OH, C(H)═N—O(C₁₋₆ alkyl), C(C₁₋₆ alkyl)═N—OH and C(C₁₋₆ alkyl)═N—O—(C₁₋₆ alkyl), and thienyl, which may itself be substituted by 1 to 3 substituents, each independently selected from halogen, cyano, nitro, C₁₋₄ alkyl, C₁₋₄ haloalkyl, C₁₋₄ alkoxy, C₁₋₄ haloalkoxy, C₁₋₄ haloalkylthio, C(H)αN—OH, C(H)═N—O(C₁₋₆ alkyl), C(C₁₋₆ alkyl)═N—OH and C(C₁₋₆ alkyl)═N—O—C₁₋₆ alkyl)

or R³⁵ is a 5-6 membered heterocyclic ring, wherein the heterocyclic ring contains 1 to 3 heteroatoms, each heteroatom independently chosen from oxygen, sulphur and nitrogen, wherein the heterocyclic ring may be substituted 1 to 3 substituents, each independently selected from halogen, cyano, nitro, C₁₋₄ alkyl, C₁₋₄ haloalkyl, C₁₋₄ alkoxy, C₁₋₄ alkylthio, C₁₋₄alkylthio, C₁₋₄ haloalkoxy, C(H)═N—O—(C₁₋₆ alkyl) and C(C₁₋₆ alkyl)═N—O—(C₁₋₆ alkyl), C₂₋₅ alkenyl, C₂₋₅ alkynyl, CHO, COOC₁-C₆ alkyl, CrC₄ alkoxy-C₁-C₄ alkyl, CrC₄ haloalkoxy-C₁-C₄ alkyl, (E)_{P}C≡CR, (E)ₙCR²⁷═CR²⁵ R²⁶, phenyl, which may itself be substituted by 1 to 3 substituents, each independently selected from halogen, cyano, nitro, C₁₋₄ alkyl, C₁₋₄ haloalkyl, C₁₋₄ alkoxy, C₁₋₄ haloalkoxy, C_{1- 4} haloalkylthio, C(H)═N—OH, C(H)═N—O(C₁₋₆ alkyl), C(C₁₋₆ alkyl)═N—OH and C(C₁₋₆ alkyl)═N—O—(C₁₋₆ alkyl), and thienyl, which may itself be substituted by 1 to 3 substituents, each independently selected from halogen, cyano, nitro, C₁₋₄ alkyl, C₁₋₄ haloalkyl, C₁₋₄ alkoxy, C₁₋₄ haloalkoxy, C₁₋₄ haloalkylthio, C(H)═N—OH, C(H)═N—O(C₁₋₆ alkyl), C(C₁₋₆ alkyl)═N—OH and C(C₁₋₆ alkyl)═N—O—(C₁₋₆ alkyl), and wherein two substituents on adjacent carbon atoms of the 5-6 membered heterocyclic ring together may form a group —CR^{35a}—CR^{35a}═CR^{35a}—CR^{35a}—, wherein each R^{35a} independently is selected from hydrogen, halogen, cyano, nitro, C₁₋₄ alkyl, C₁₋₄ haloalkyl, C₁₋₄ alkoxy, C₁₋₄ haloalkoxy, C₁₋₄ haloalkylthio, C(H)═N—OH, C(H)═N—O(C₁₋₆ alkyl), C(C₁₋₆ alkyl)═N—OH and C(C₁₋₆ alkyl)═N—O—(C₁₋₆ alkyl);

or R³⁵ is an aliphatic saturated or unsaturated group containing 3 to 13 carbon atoms and at least one silicon atom, wherein the aliphatic group may contain 1 to 3 heteroatoms, each heteroatom independently selected from oxygen, nitrogen and sulphur, and wherein the aliphatic group may be substituted by 1 to 4 independently selected halogen atoms;

or R³⁵ is (CR^{a} R^{b})ₘ—Cy-(CR^{c} R^{d})ₙ-A;

or R³⁵ is C₁₋₆ alkoxy, C₁₋₆ haloalkoxy, C₂₋₆ alkenyloxy, C₂₋₆ haloalkenyloxy, C₂₋₆ alkinyloxy, C₃₋₆ cycloalkyloxy, C₁₋₄ alkyl-C₃₋₇ cycloalkyloxy, C₅₋₇ cycloalkenyloxy or C₁₋₄ alkyl-C₅₋₇ cycloalkenyloxy;

E is C₁₋₄ alkylene;
p is 0 or 1;

R²⁴ is hydrogen, halogen, C₁₋₄ alkyl, C₁₋₄ haloalkyl, C₁₋₄ alkoxy (C₁₋₄) alkyl, C₁₋₄ haloalkoxy (C₁₋₄) alkyl or Si(C₁₋₄ alkyl)₃;

R²⁵ and R²⁶ are each, independently, hydrogen, halogen, C₁₋₄ alkyl or C₁₋₄ haloalkyl;

R²⁴ is hydrogen, C₁₋₄ alkyl or C₁₋₄ haloalkyl;

R^{a}, R^{b}, R^{c} and R^{d} are each, independently, hydrogen or a C₁₋₄ alkyl group, which may substituted by 1 to 6 substituents, each substituent independently selected from halogen, hydroxy, cyano, carboxyl, methoxycarbonyl, ethoxycarbonyl, methoxy, ethoxy, methylsulfonyl, ethylsulfonyl, difluoromethoxy, trifluoromethoxy, trifluoromethylthio and trifluorothiomethoxy ;

Cy is a carbocyclic or heterocyclic 3-7 membered ring, which may be saturated, unsaturated or aromatic and which may contain a silicon atom as a ring member, wherein (CR^{a} R^{b})ₘ and (CR^{c} R^{d})ₙ may be bound either to the same carbon or silicon atom of Cy or to different atoms separated by 1, 2 or 3 ring members, wherein the carbocyclic or heterocyclic 3-7 membered ring may substituted by 1 to 6 substituents, each substituent independently selected from halogen, C₁₋₄ alkyl, C₂₋₄ alkenyl, C₁₋₄ haloalkyl, C₁₋₄alkoxy and halo-C₁₋₄ alkoxy;

A₁ is Si(Oₚ₁E¹)(O≤E³) and provided that Cy contains a silicon atom as a ring member then A₁ may also be hydrogen;

E¹ and E² are independently methyl or ethyl;

E³ is a C₁₋₄ alkyl or a C₂₋₄ alkenyl group, which may be interrupted by one heteroatom selected from O, S and N, and wherein the C₁₋₄ alkyl or C₂₋₄ alkenyl group may be substituted by 1 to 3 independently selected halogen atoms;

m and n are each independently 0, 1, 2 or 3;
p₁, q and s are each independently 0 or 1;
R⁶, R⁷, R⁸, R⁹, R¹⁰, R¹¹ and R^{11a} are each, independently, hydrogen, halogen, cyano, nitro, C₁₋₄ alkyl, C₁₋₄ haloalkyl, C₁₋₄ alkoxy, C₁₋₄ haloalkoxy, C₁₋₄ thioalkyl or C₁₋₄ thiohaloalkyl;

R¹², R¹³, R¹⁴, R¹⁵ and R¹⁶ are each, independently, hydrogen, halogen, cyano, nitro, C₁₋₄ alkyl, C(O)CH₃, C₁₋₄ haloalkyl, C₁₋₄ alkoxy, C₁₋₄ haloalkoxy, C₁₋₄ thioalkyl, C₁₋₄ thiohaloalkyl, hydroxymethyl or C₁₋₄ alkoxymethyl;

T is a single or a double bond; and

A is O, N(R¹⁷), S or (CR18R19)(CR²⁰_{R}²¹)ₘ₁ (CR²²R²³)ₙ₁;

R¹⁷ is hydrogen, C₁₋₄ alkyl, formyl, C₁₋₄ alkoxy(C₁₋₄)alkyl, C(50 O)C₁₋₄ alkyl, which may be substituted by halogen or C₁₋₄-alkoxy, or C(═O)O—C₁₋₆ alkyl, which may be substituted by halogen, C₁₋₄ alkoxy or CN;

R¹⁸, R¹⁹, R²⁰, R²¹, R²² and R²³ are each independently hydrogen, halogen, hydroxy, C₁₋₄ alkoxy, C₁₋₆ alkyl, which may be substituted by 1 to 3 substituents selected from halogen, hydroxy, ═O, C₁₋₄ alkoxy, O—C(O)—C₁₋₄ alkyl, phenyl, naphthyl, anthracyl, fluorenyl, indanyl or a 3-7 membered carbocyclic ring (which itself may be substituted by 1 to 3 methyl groups), C₁₋₆ alkenyl, which may be substituted by 1 to 3 substituents selected from halogen, hydroxy, ═O, C₁₋₄ alkoxy, O—C(O)—C₁₋₄ alkyl, phenyl, naphthyl, anthracyl, fluorenyl, indanyl or a 3-7 membered carbocyclic ring (which itself may be substituted by 1 to 3 methyl groups), or a 3-7 membered carbocyclic ring, which may contain 1 heteroatom selected from nitrogen and oxygen, and wherein the 3-7 membered carbocyclic ring may be substituted by 1 to 3 methyl groups;

or R¹⁸, R¹⁹ together with the carbon atom to which they are attached form a carbonyl-group, a 3-5 membered carbocyclic ring, which may be substituted by 1 to 3 methyl groups, C₁₋₆ alkylidene, which may be substituted by 1 to 3 methyl groups, or C₃₋₆ cycloalkylidene, which may be substituted by 1 to 3 methyl groups;

m₁ is 0 or 1;

n₁ is 0 or 1;

R^{12a} is a C₁-C₄ alkyl, C₂-C₄ alkenyl or C₂-C₄ alkynyl group, which may be substituted by 1 to 6 substituents, each substituent independently selected from halogen, hydroxy, cyano, C₁₋₄ alkoxycarbonyl, formyl, nitro, C₁-C₄ alkoxy, C₁-C₄ haloalkoxy, C₁-C₄ alkylthio, C₁-C₄ haloalkylthio, HC(OR²⁸)═N— and R²⁹R³⁰NN═C(H)—;

R²⁸, R²⁹ and R³⁰ independently of one another are hydrogen or C₁-C₄ alkyl;

R^{12b} is a C₁-C₆ alkyl group, which may be substituted by 1 to 6 substituents, each substituent independently selected from halogen, hydroxy, cyano,

C₁₋₄ alkoxycarbonyl, formyl, nitro, C₁-C₄ alkoxy, C₁-C₄ haloalkoxy,
C₁-C₄ alkylthio, C₁-C₄ haloalkylthio, HC(OR³¹)═N— and R³²R³³NN═C(H)—;
R³¹, R³² and R²³ independently of one another are hydrogen or C₁-C₄ alkyl;
R^{12c} is hydrogen or halogen; and tautomers/isomers/enantiomers of these compounds.

Preferably, Q1 is selected from the group consisting of Q39, Q40, Q41, Q42, Q43 and Q44, wherein Q39, Q40,Q41, Q42, Q43 and Q44 Q39, Q40,are defined below

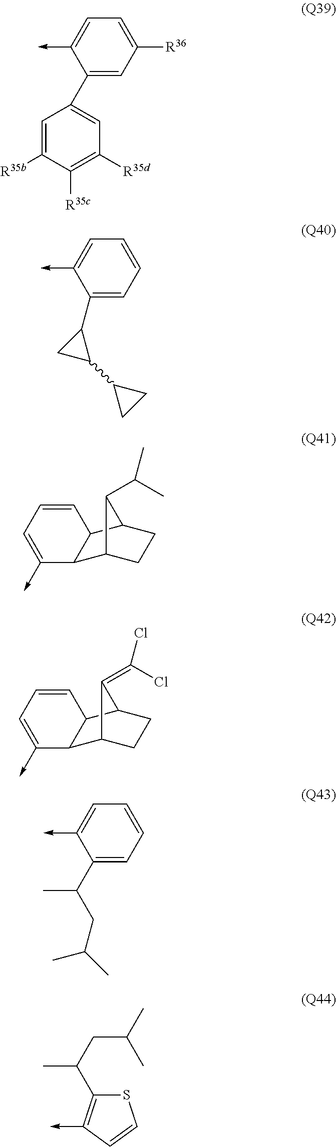

In a first preferred embodiment of the present invention, Q is a group of formula Q39

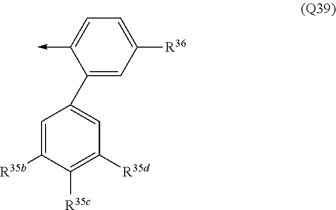

wherein R³⁶, R^{35b}, R^{35c} and R^{35d} are each, independently, selected from the group consisting of hydrogen and halogen, said halogen is especially chlorine or fluorine. In one preferred aspect, R^{35b}, R^{35c} and R^{35d} are F and R³⁶ is H. In another preferred aspect, R³⁶ is F, R^{35b} and R^{35c} are Cl and R^{35d} is H.

In a second preferred embodiment of the present invention, Q is a group of formula, Q is a group of formula Q40

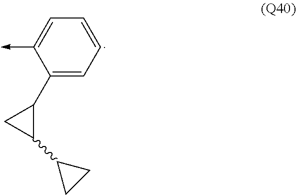

In a third preferred embodiment of the present invention, Q is a group of formula, Q is a group of formula Q41

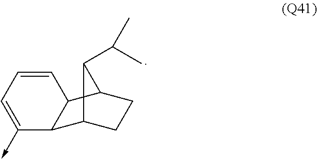

In a fourth preferred embodiment of the present invention, Q is a group of formula, Q is a group of formula Q42

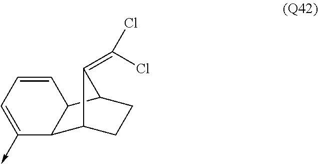

In a fifth preferred embodiment of the present invention, Q is a group of formula, Q is a group of formula Q43

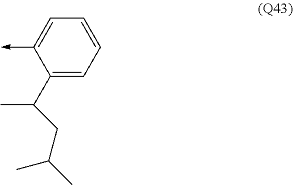

In a sixth preferred embodiment of the present invention, Q is a group of formula, Q is a group of formula Q44

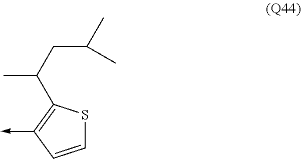

In a seventh preferred embodiment of the present invention, R¹ is CF₂H, R² is CH₃, R⁴is H and R⁵ is CH₃ in formula (I), A* is absent and R³ is H in formula (II).

In a eighth preferred embodiment of the present invention, R¹ is CF₂Cl, R² is CH₃, R⁴is H and R⁵ is CH₃ in formula (I), A* is absent and R³ is H in formula (II).

In an ninth preferred embodiment of the present invention, R¹ is CF₃, R² is CH₃, R⁴is H and R⁵ is CH₃ in formula (I), A* is absent and R³ is H in formula (II).

Each of the seventh to ninth embodiments above can be combined with each of the first to sixth embodiments above.

In one embodiment of the present invention, the process for the manufacture of compound (III) further comprises a step of reductive dehalogenation, where R¹ is R¹ is CF₂Cl before the reductive dehalogenation step and R¹ is R^{1″} is CHF₂ after the reductive dehalogenation step. In one aspect, in the compound of formula (I), R¹ is R¹ is CClF₂, compound (I) is reacted with the compound of formula (II) to form the compound of formula (III) in which R¹ is R¹ is CClF₂; subsequently, the compound of formula (III) is submitted to a step of reductive dehalogenation to form the compound of formula (III) in which R¹ is R^{1″} is CHF₂. For such a step, the reaction conditions and reagents are described WO2012010692, which is hereby incorporated by reference for all purposes, as disclosed for the reductive dehalogenation for the compound of formula (II) therein.

In another embodiment of the present invention, the process for the manufacture of a compound of formula (III) comprises a step of reductive dehalogenation, where R¹ is R¹ is CF₂Cl before the reductive dehalogenation step and R¹ is R^{1″} is CHF₂ after the reductive dehalogenation step, wherein in the compound of formula (I) R¹ is R^{1′} is CClF₂, the compound of formula (I) is submitted to a step of reductive dehalogenation to form the compound of formula (I) in which R¹ is R^{1″} is CHF₂. Subsequently, the compound (I) in which R¹ is R^{1″} is CHF₂ is reacted with the compound of formula (II) to form the compound of formula (III) in which R¹ is CHF₂. For such a step, the reaction conditions and reagents can be taken from WO2012010692, which is hereby incorporated by reference for all purposes, as disclosed for the reductive dehalogenation for the compound of formula (II) therein, or alternatively from WO2009021987, which is hereby incorporated by reference for all purposes, as disclosed for the reductive dehalogenation for the esterified ketene adduct of RCFClC(O)Cl disclosed therein.

In a preferred embodiment according to the present invention, the step of reacting (I) and (II) to obtain (III) is performed in the presence of at least one oxidation agent, which preferably is oxygen. The oxidation agent is, for example, selected from the group consisting of oxygen, an organic or inorganic peroxide, for example H₂O₂, anions of halogenoxgyenacids, such as bromate or hypochlorite, MnO₄⁻, Cr₂O₇²⁻, Cr(VI) oxide, elemental halogens. Preferably, the reaction is performed in the presence of oxygen. The amount of oxygen or oxidation agent is equal to or more than 1 eq in relation to compound (I). Preferably, an oxygen atmosphere or oxygen containing atmosphere, which comprises at least 10 vol % O₂, is present.

In one aspect, the reaction is performed in a sealed vessel in an oxygen atmosphere or oxygen containing atmosphere.

In another preferred embodiment, the step of reacting (I) and (II) to obtain (III) is performed in the presence of at least one catalyst. Often, the at least one catalyst comprises at least one metal compound, wherein the at least one metal compound is a metal salt or a metal complex. Preferably, the at least one metal compound comprises at least one metal ion or metal atom selected from the group consisting of transition metals. In one aspect, the at least metal compound is selected from the group consisting of transition metal halides, transition metal cyanates or transition metal acetates. Preferably, the at least one metal compound is selected from the group consisting of CuBr, CuCN, CuCl₂ and Pd(OAc)₂. In another aspect, the at least metal compound is selected from the group consisting of transition metal complexes. More preferably, the transition metal complex is selected from the group consisting of palladium and ruthenium complexes, in particular PdCl₂(dppf) or Pd₂(dba)₃.

In one embodiment, at least one oxidation agent, preferably oxygen and optionally at least one other oxidation agent, more preferably oxygen as single oxidaton agent, and at least one catalyst are present in the step of reacting (I) and (II) to obtain (III). According to this aspect, R⁵ is selected from the group consisting of C₁-C₁₂-alkyl, C₂-C₆ alkenyl, cycloalkyl, aryl, heteroaryl, aralkyl, each of which are optionally substituted, preferably R⁵ is an optionally substituted C₁-C₄ alkyl group or cycloalkyl group, more preferably R⁵ is selected from the group consisting of methyl, ethyl, i-propyl, n-propyl, n-butyl, tert-butyl, iso-butyl and sec-butyl, wherein methyl is preferred. The C₁-C₄ alkyl group, preferably methyl, optionally is substituted by zero, one or more halogen atoms, independently selected from the group consisting of Cl, Br, F, and I, wherein Br, Cl and F are preferred. In this embodiment, the optionally substituted methyl group includes CF₃, CCl₃, CBr₃ and CI₃.

The catalyst, when present, often is present in an amount of equal to or greater than 0.1 mol %, related to the amount of formula (I). More preferably, the at least one catalyst is present in an amount of equal to or more than 1 mol %. Even more preferably, the at least one catalyst is present in an amount of equal to or more than 5 mol %. An amount of equal to or more than 10 mol % is also suitable. Further, the at least one catalyst, when present, often is present in an amount of equal to or less than 50 mol %, preferably equal to or less than 40 mol % and more preferably equal to or lower than 30 mol %.

Often, when at least one catalyst is present in the step of the step of reacting (I) and (II) to obtain (III), a base is present, which is generally not (II). Often, the base which is generally not (II) is selected from the group consisting of acyclic tertiary amines, such as trimethylamine, triethylamine, diisopropylethylamine, tert-butyldimethylamine or ethyldicyclohexylamine, the cyclic tertiary amines, such as N-methylpyrrolidine, N-methylpiperidine, N-methylmorpholine, N,N′-dimethylpiperazine, pyridine, collidine, lutidine or 4-dimethylaminopyridine, or bicyclic amines, such as diazabicycloundecene (DBU) or diazabicyclononene (DBN). Particularly preferred is pyridine. Generally, an excess of (II) can also be employed as base. The base is often present in an amount of equal to or more than 1 eq, in relation to the amount (I), but may also be present in an amount of equal to or more than 2 eq, or even equal to or more than 3 eq. While the base can also be employed as a solvent for the reaction, the amount of base generally is equal to or lower than 10 eq, preferably equal to or lower than 6 eq, and even more preferably equal to or lower than 4 eq.

In the process according to the present invention, the temperature at which the step of reacting (I) and (II) to obtain (III) is performed is often at a temperature which generally is equal to or greater than 20° C., preferably equal to or greater than 30° C. and more preferably equal to or greater than 40° C. Often, the temperature is equal to or less than 150° C., preferably equal to or less than 140° C. and more preferably equal to or less than 130° C.

In one aspect of the process according to the present invention, the step of reacting (I) and (II) to obtain (III) is performed in the presence of a solvent. Appropriate solvents suitable for reacting the compound of the formula (I) and (II) to obtain (III) are, for example, protic polar solvents, such as aliphatic alcohols having preferably 1 to 4 carbon atoms, especially methanol, ethanol, n-propanol, isopropanol, n-butanol, isobutanol or tert-butanol, nonpolar aprotic solvents, e.g. aromatic hydrocarbons, such as benzene, toluene, xylenes, mesitylene, cumene, chlorobenzene, nitrobenzene or tert-butylbenzene, aprotic polar solvents, such as cyclic or acyclic ethers, especially diethyl ether, tert-butyl methyl ether (MTBE), cyclopentyl methyl ether, tetrahydrofuran (THF) or dioxane, cyclic or acyclic amides, especially dimethylformamide, dimethylacetamide, N-methylpyrrolidone, ureas, such as N,N′-dimethyl-N,N′-ethyleneurea (DMEU), N,N′-dimethyl-N,N′-propyleneurea (DMPU) or tetramethylurea, or aliphatic nitriles, especially acetonitrile or propionitrile, or mixtures of the solvents mentioned above. Particularly preferred are aromatic hydrocarbons, such as xylenes, toluene or benzene.

The reaction product can be isolated according to common procedures, which can include crystallization, washing, distillation, drying, concentration at elevated temperature and/or subambient pressure, chromatography, filtration and/or salt formation.

The invention concerns further a process for the manufacture of an agrochemically or pharmaceutically active compound, which comprises the process according to the present invention, which comprises the step of reacting a compound of formula (I) and a compound of formula (II). Preferably, the active compound is an agrochemically active compound. More preferably, the agrochemically active compound is a SDHI fungicide, a derivative or precursor thereof. Even more preferably, the SDHI fungicide is selected from the group consisting of Benzovindiflupyr, Bixafen, Fluxapyroxad, Furametpyr, Isopyrazam, Penflufen, Penthiopyrad, Sedaxane and compound Ib as disclosed in WO2013/120860 and its isomers and mixtures of isomers.

The invention further concerns a pharmaceutical or agrochemical formulation comprising a pharmaceutical or agrochemically active compound which was obtained in a process comprising the process according to the present invention which comprises the step of reacting a compound of formula (I) and a compound of formula (II) to obtain the compound of formula (III).

Another object of the present invention is the use of a compound of formula (I) for the manufacture of an agrochemically or pharmaceutically active compound, wherein (I) is reacted with a compound of formula (II).

The compound (I) in the process according to the present invention can be manufactures, for example, by reacting a compound of formula (VI)

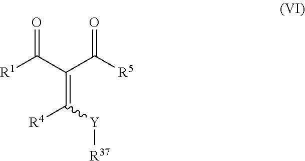

with a compound of formula (VII), (VIII) or (IX).

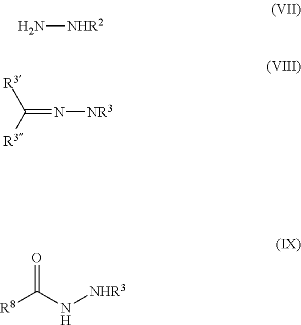

R² is defined as above, and R³, R^{2′} and R^{2″} independently from each other in (VIII) are selected from the group consisting of C₁-C₁₂-alkyl, C₂-C₆ alkenyl, cycloalkyl, aryl, heteroaryl, aralkyl, each of which are optionally substituted. The invention thus also concerns a process for the manufacture of (III), which further comprises the step of reacting a compound of formula (VI) with a compound selected from the group consisting of (VII), (VIII) and (IX).

In one embodiment of the present invention, the process for manufacturing a compound of formula (III) which comprises a step of reacting a compound of formula (I) and (II)further comprises a step of manufacturing the compound of formula (VI). In one aspect, the step of manufacturing compound (VI) comprises reacting a compound of formula (IVa) with a compound of formula (Va) or (Vb) to obtain compound (VI), wherein X″ is selected from F, Cl and Br, preferably F and Cl, Y is selected from O, S and NR³⁸, preferably NR³⁸ and O, and R³⁷ and R³⁸ independently are selected from the group consisting of C₁-C₁₂-alkyl, more preferably C₁-C₄-alkyl, aralkyl, aryl, heteroaryl and cycloalkyl, all of which are optionally substituted, and wherein R¹, R⁴ and R⁵ are defined as above.

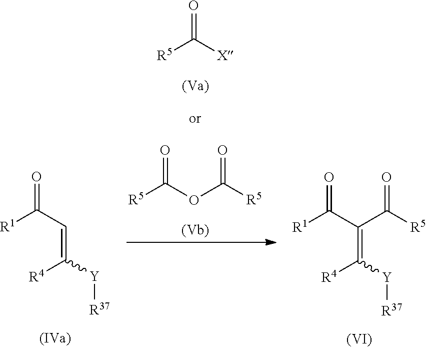

In another aspect, the step of manufacturing compound (VI) comprises reacting a compound of formula (IVb) with a compound of formula (Vc) or (Vd) to obtain compound (VI).

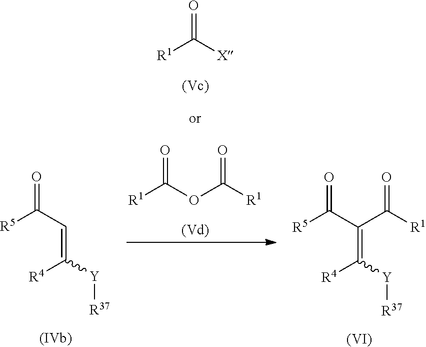

R¹, R⁴, Y, R³⁷ and R⁵ are defined as already disclosed above for the compound of formula (I).

The compounds of formula Va and Vc are known to be carboxylic acid halides. Many compounds falling under the formula Va and Vc are well established and commercially available. The manufacture of difluoroacetyl fluoride is, for example, disclosed in EP694523 and U.S. Pat. No. 5,905,169 which are hereby incorporated by reference for all purposes. The manufacture of difluorochloroacetyl chloride is, for example, disclosed in U.S. Pat. No. 5,545,298 or U.S. Pat. No. 5,569,782, which are hereby incorporated by reference for all purposes, as well as the manufacture of trifluoroacetylchloride. The manufacture of halogenated carboxylic acid anhydrides such as Vb and Vd is known, for example, from WO2014195929, which is hereby incorporated by reference for all purposes. In one aspect, a compound of formula (IVe) R¹—C(O)—O—C(O)—R² can also be used for the manufacture of compound (I). The manufacture of mixed anhydrides is described, for example, in WO200117939, which is hereby incorporated by reference for all purposes, and can by also applied to compounds of formula (IVe).

In one aspect, the compound of formula (I) is further used as crude reaction product when manufactured from (IVa) or (IVb), for example in the manufacture of a formula of compound (III). The reaction product may also be separated from any salts produced in the reaction, for example by filtration, washing, decanting or spinnning, and then is reacted further without further purification. When desired, the crude reaction mixture can also be purified, for example by distillation, crystallization, chromatography or distillation.

In one embodiment, the process for the manufacture of a compound of formula (III) from a compound of formula (I) further comprises the step of manufacturing the compound of formula (IVa) or (IVb), with Y =NR³⁸, from a compound of formula (VIa) or (VIb)

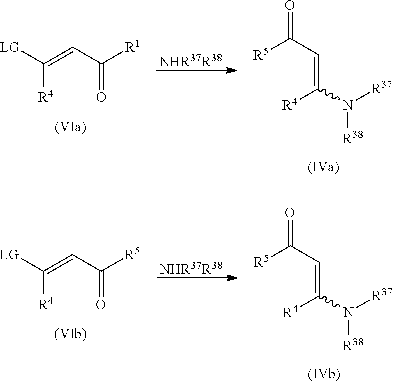

LG is a suitable leaving group, preferably LG is alkoxy R³⁹O— or aryloxy ArO—. R³⁹ is selected from the group consisting of C₁-C₁₂-alkyl or C₃-C₁₀-cycloalkyl group, each of which is optionally substituted. Preferably, R³⁹ is methyl, ethyl, n- or i-propyl or i-, n- or tert-butyl, wherein methyl and ethyl are most preferred R³⁹. The compounds of formula (VIb) and (VIa) can be prepared, for example by reacting ethylvinylether and the corresponding acid halide.

R³⁷ and R³⁸ in the step of manufacturing (IVa) or (IVb) from a compound of formula (VIa) or (VIb) have the same meaning as for the compound of formula (I). In a preferred aspect, R³⁷ and R³⁸ are Methyl or Ethyl.

The process according to the present invention allow for efficient syntheses of agrochemical and pharmaceutical compounds. The present processes for obtaining agrochemically or pharmaceutically active ingredients or intermediates thereof generally comprise less steps than currently available processes, allowing for economically and ecologically advantageous manufacture. Often, the process steps display good to excellent yields and selectivities.

Should the disclosure of any patents, patent applications, and publications which are incorporated herein by reference conflict with the description of the present application to the extent that it may render a term unclear, the present description shall take precedence.

The following examples are intended to further explain the invention without limiting it.

### EXAMPLES

Difluoroacetylfluoride (DFAF) can be obtained from commercial sources, or manufactured according to the publications cited in the description. 3′,4′-dichloro-5-fluorobiphenyl-2-amine, 3′,4′,5′-trifluorobiphenyl-2-amine and 2-(bi(cyclopropan)-2-yl)aniline can be obtained from commercial sources.

### Example 1: 4-(dimethylamino)but-3-en-2-one

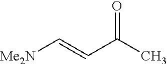

4-ethoxy-3-buten-2-one is prepared by reacting ethylvinylether and acetylchloride. The product is mixed with dichloromethane and cooled to —5° C. 40% v/v of aqueous dimethylamine (1.1 eq) is added, the mixture is stirred for 10 minutes at −5° C., warmed to room temperature by removing the ice bath and stirred for one hour at room temperature. The mixture is washed with brine, dried over NaSO₄ and the volatiles are removed in vacuo. The product is used without further purification.

### Example 2: 3-((dimethylamino)methylene)-1,1-difluoropentane-2,4-dione

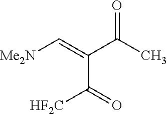

20 g of the product of example 1 is mixed with 140 mL dichloromethane. Pyridine (1.05 eq) is added and the mixture is cooled to −15° C. Difluoroacetylfluoride (DFAF, 1.05 eq) in dichloromethane is added over a period of 60 minutes. The mixture is stirred at −15° C. for 20 minutes, slowly warmed to room temperature and stirred at room temperature for another 60 minutes. The mixture is diluted with water (80 mL) mixed thoroughly, the phases are separated and the aqueous phase extracted twice with dichloromethane. The combined extracts are concentrated in vacuo to remove the volatiles.

### Example 3: 1-(3-(difluoromethyl)-1-methyl-1H-pyrazol-4-yl)lethanone

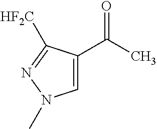

Monomethylhydrazine (40% v/v in water, 1.05 eq) and 15 g of the product of example 2 are mixed with 80 mL acetonitrile. The mixture is stirred at room temperature for 14 hours. The volatiles are removed in vacuo. The aqueous phase is extracted twice with ethyl acetate, the combined extracts are concentrated in vacuo to remove the volatiles.

### Example 4: Bixafen (N-(3′,4′-dichloro-5-fluorobiphenyl-2-yl)-3-(difluoromethyl)-1-methyl-1H-pyrazole-4-carboxamide)

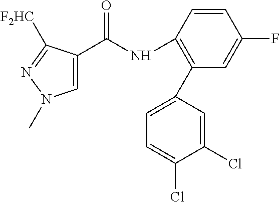 [SMILES: Cn1cc(C(=O)Nc2ccc(F)cc2-c2ccc(Cl)c(Cl)c2)c(C(F)F)n1]

The product of example 3 (5.0g, 18 mmol) and 3′,4′-dichloro-5-fluorobiphenyl-2-amine (5.52 g, 21.6 mmol) are dissolved in 50 ml xylene. To this solution pyridine (5 ml, 2 eq) and 10 mol % CuBr is heated for 12 h at 130° C. The mixture is cooled to 20° Cm filtered, washed with water, dried over Na₂O₄ and the volatiles are evaporated. The residue is triturated with cold water to yield a gray suspension. Solids are filtered, washed with water and dried yielding crude Bixafen.

### Example 5: Fluxapyroxad (3-(difluoromethyl)-1-methyl-N-(3′,4′,5′-trifluorobiphenyl-2-yl)-1H-pyrazole-4-carboxamide)

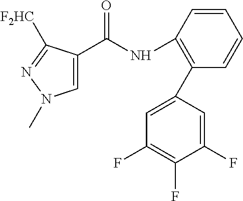 [SMILES: Cn1cc(C(=O)Nc2ccccc2-c2cc(F)c(F)c(F)c2)c(C(F)F)n1]

Fluxapyroxad is obtained using the procedure of example 4, wherein 3′,4′,5′-trifluorobiphenyl-2-amine is used instead of 3′,4′-dichloro-5-fluorobiphenyl-2-amine.

### Example 6: Sedaxane (N-(2-(bi(cyclopropan)-2-yl)phenyl)-3-(difluoromethyl)-1-methyl-1H-pyrazole-4-carboxamide)

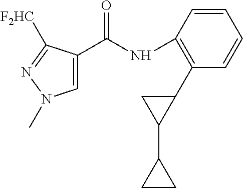 [SMILES: Cn1cc(C(=O)Nc2ccccc2C2CC2C2CC2)c(C(F)F)n1]

Sedaxane is obtained using the procedure of example 4, wherein 2-(bi(cyclopropan)-2-yl)aniline is used instead of 3′,4′-dichloro-5-fluorobiphenyl-2-amine.

1. A process for the manufacture of carboxamides, the process comprising the step of reacting a pyrazole compound of the formula (I) 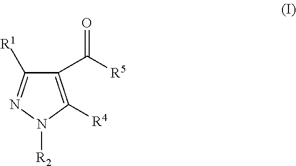
with a compound of the formula (II) NR³H(A*)Q to obtain a compound of the formula (III) 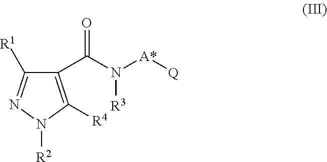
wherein R¹ is selected from the group consisting of H, R′, X′, CN, COOR′, OR′, SR′ and C(O)NR′₂, wherein R′ is selected independently from the group consisting of hydrogen, CN, C₁-C₁₂-alkyl, C₁-C₁₂-alkyl, C₂-C₆ alkenyl, aryl, cycloalkyl, aralkyl and heteroaryl, each of which is optionally substituted, and X′ is selected from the group consisting of F, Cl, Br and I;
wherein R² is selected from the group consisting of optionally substituted C₁-C₁₂-alkyl, C₂-C₆ alkenyl, cycloalkyl, aryl, heteroaryl, and aralkyl; wherein R³ is selected from the group consisting of H, optionally substituted C₁-C₁₂-alkyl, C₂-C₆ alkenyl [[or]] and C₃-C₈-cycloalkyl group;
wherein R⁴ is selected from the group consisting of H, X′, COOR′, OR′, SR′, and C(O)NR′₂, wherein R′ and X′ are as defined above;
wherein R⁵ is selected from the group consisting of C₁-C₁₂-alkyl, C₂-C₆ alkenyl, cycloalkyl, aryl, heteroaryl, and aralkyl, each of which is optionally substituted;
wherein A* is absent or an optionally substituted C₁-C₄-alkylene group;
wherein Q is an optionally substituted aryl or heteroaryl group.

2. The process according to claim 1, wherein R¹ is selected from the group consisting of CF₂Cl, CF₂H, CFCl₂, CFClH, CF₂Br, CCl₃, CF₃, CBr₃, and CI₃.

3. The process according to claim 1, wherein R⁵ is an alkyl or cycloalkyl residue, each of which is optionally substituted.

4. The process according to claim 1, wherein R⁵ is an optionally substituted C₁-C₄-alkyl group.

5. The process according to claim 1, wherein R² is H or an optionally substituted C₁-C₄-alkyl group.

6.The process according to claim 1, wherein Q is selected from the group consisting of Q39, Q40, Q41, Q42, Q43 and Q44 wherein Q39, Q40,Q41, Q42, Q43 and Q44 are defined below 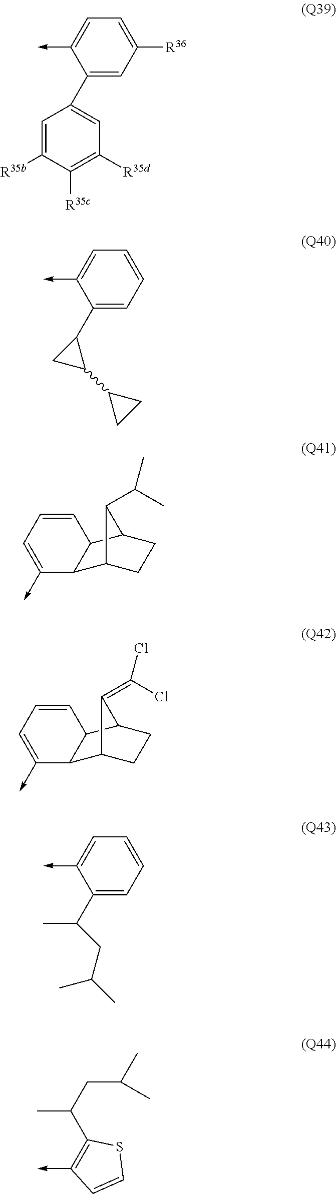
wherein R³⁶, R^{35b}, R^{35c} and R^{35d} are each, independently, selected from the group consisting of hydrogen and halogen.

7. The process according to claim 1, wherein the step of reacting (I) and (II) to obtain (III) is performed in the presence of at least one oxidation agent.

8. The process according to claim 1, wherein the step of reacting (I) and (II) to obtain (III) is performed in the presence of at least one catalyst.

9. The process according to claim 8, wherein the at least one catalyst comprises at least one metal compound, wherein the at least one metal compound is a metal salt or a metal complex.

10. The process according to claim 9, wherein the at least one metal compound comprises at least one metal ion or metal atom selected from the group consisting of transition metals.

11. The process according to claim 9, wherein the at least metal compound is selected from the group consisting of transition metal halides, transition metal cyanates and transition metal acetates.

12. The process according to claim 9, wherein the at least one metal compound is selected from the group consisting of CuBr, CuCN, CuCl₂ and Pd(OAc)₂.

13. The process according to claim 12, wherein the at least metal compound is selected from the group consisting of transition metal complexes.

14. The process according to claim 13, wherein the transition metal complex is selected from the group consisting of palladium and ruthenium complexes.

15. A process for the manufacture of an agrochemically or pharmaceutically active compound, which comprises the process of claim 1.

16. The process according to claim 5, wherein R² is optionally substituted methyl.

17. The process according to claim 7, wherein the at least one oxidation agent is oxygen.

18. The process according to claim 14, wherein the transition metal complex is PdCl₂(dppf) or Pd₂(dba)₃.
